(19) United States
(12) Patent Application Publication
(10) Pub. No.: US 2022/0313356 A1
(43) Pub. Date: Oct. 6, 2022

(54) HIGH-FREQUENCY TREATMENT TOOL AND METHOD FOR MANIPULATING HIGH-FREQUENCY TREATMENT TOOL

(71) Applicant: OLYMPUS CORPORATION, Tokyo (JP)

(72) Inventors: Yoshinori HIGUCHI, Tokyo (JP); Chika MIYAJIMA, Tokyo (JP); Satoko KATO, Tokyo (JP)

(73) Assignee: OLYMPUS CORPORATION, Tokyo (JP)

(21) Appl. No.: 17/839,735

(22) Filed: Jun. 14, 2022

Related U.S. Application Data
(63) Continuation of application No. PCT/JP2019/049127, filed on Dec. 16, 2019.

(51) Int. Cl. A61B 18/14

(52) U.S. Cl. CPC A61B 18/1492; A61B 2018/00601

(57) ABSTRACT

A high-frequency treatment tool includes: a tubular sheath; a cap member fixed to a distal end of the sheath; and an electrode part provided so as to be relatively movable in a longitudinal direction of the sheath and relatively rotatable about a longitudinal axis of the sheath with respect to the cap member. The electrode part and the cap member are relatively rotated about the longitudinal axis of the sheath while pressing biological tissue stuck to the electrode part against the cap member to separate the biological tissue from the electrode part.

### CROSS-REFERENCE TO RELATED APPLICATIONS

This is a continuation of International Application PCT/JP2019/049127 which is hereby incorporated by reference herein in its entirety.

### TECHNICAL FIELD

The present invention relates to a high-frequency treatment tool and a method for manipulating a high-frequency treatment tool.

### BACKGROUND ART

A known high-frequency treatment tool in the related art endoscopically cuts biological tissue, such as a mucous membrane (for example, see PTL 1). The high-frequency treatment tool disclosed in PTL 1 includes a rod-like electrode projecting in the longitudinal direction from the distal end of a sheath. The high-frequency treatment tool disclosed in PTL 1 cuts and cauterizes biological tissue by bringing the electrode energized with a high-frequency current into contact with the biological tissue.

In the high-frequency treatment tool disclosed in PTL 1, when biological tissue is cut and cauterized, a burnt piece of the cut biological tissue sticks to the electrode, which degrades the cutting performance. Therefore, when a burnt piece of biological tissue is stuck to the electrode, the high-frequency treatment tool is temporarily removed from an endoscope channel, the burnt piece of biological tissue is removed from the electrode, and then the high-frequency treatment tool is inserted again into the endoscope channel to perform treatment.

### CITATION LIST

### Patent Literature

### {PTL 1}

PCT International Publication No. WO 2014/042039

### SUMMARY OF INVENTION

A first aspect of the present invention is a high-frequency treatment tool including: a tubular sheath; a cap member fixed to a distal end of the sheath; and an electrode part provided so as to be relatively movable in a longitudinal direction of the sheath and relatively rotatable about a longitudinal axis of the sheath with respect to the cap member. The electrode part and the cap member are relatively rotated about the longitudinal axis of the sheath while pressing biological tissue stuck to the electrode part against the cap member to separate the biological tissue from the electrode part.

A second aspect of the present invention is a high-frequency treatment tool including: a tubular sheath; a cap member fixed to a distal end of the sheath; and a rod-like member extending along a longitudinal axis of the sheath through the cap member and provided so as to be relatively movable along the longitudinal axis of the sheath and relatively rotatable about the longitudinal axis of the sheath with respect to the cap member. The rod-like member includes, at a distal end thereof, at least one electrode extending in a direction intersecting a longitudinal axis of the rod-like member. The cap member has at least one step in a direction facing the electrode. The step extends in the direction intersecting the longitudinal axis of the rod-like member. The rod-like member and the sheath are configured to relatively move along the longitudinal axis of the sheath and are configured to relatively rotate about the longitudinal axis of the sheath.

A third aspect of the present invention is a method for manipulating a high-frequency treatment tool, the method including: in a state in which the high-frequency treatment tool including an electrode part projecting in a longitudinal direction of a sheath through a cap member fixed to a distal end of the sheath is inserted through a living body, relatively moving the electrode part and the sheath in a direction in which the electrode part is drawn into the sheath to press biological tissue stuck to the electrode part against the cap member; and relatively rotating the electrode part and the cap member about a longitudinal axis of the sheath while pressing the biological tissue against the cap member to separate the biological tissue from the electrode part.

### BRIEF DESCRIPTION OF DRAWINGS

FIG. 1 shows that the mucous membrane in the body is cut and cauterized with a high-frequency treatment tool according to an embodiment of the present invention.

FIG. 2 shows the overall configuration of the high-frequency treatment tool in FIG. 1.

FIG. 3 is a plan view of a handle of the high-frequency treatment tool, as viewed in the direction parallel to the plane of the drawing of FIG. 2.

FIG. 4 is a perspective view showing the distal end of a sheath in FIG. 2.

FIG. 5 is a perspective view showing an insulating chip in FIG. 4.

FIG. 6 is a vertical cross section showing the distal end of the sheath and a knife part in FIG. 2.

FIG. 7 is a perspective view showing an electrode part and an electrical insulator in FIG. 6.

FIG. 8 is a plan view of the electrode part and the electrical insulator in FIG. 7, as viewed from the back.

FIG. 9 is a plan view for explaining relative movement and rotation of the knife part and the sheath in FIG. 2.

FIG. 10 is a plan view showing an example state in which a burnt piece of biological tissue is stuck to a first electrode and a second electrode in FIG. 9.

FIG. 11 is a flowchart explaining a method for removing a burnt piece of biological tissue with the high-frequency treatment tool according to the embodiment of the present invention.

FIG. 12 is a perspective view showing a state in which the biological tissue is pressed against the insulating chip by drawing the first electrode in FIG. 10 into the sheath.

FIG. 13 is a perspective view showing an example state in which the electrode part and the sheath are relatively rotated while the burnt piece of biological tissue is pressed against the insulating chip.

FIG. 14 is a perspective view showing an example insulating chip having multiple grooves in one surface.

FIG. 15 is a perspective view showing an example insulating chip having one projection on one surface.

FIG. 16 is a perspective view showing an example insulating chip having multiple projections on one surface.

FIG. 17 is a perspective view showing an example state in which a burnt piece of biological tissue is stuck to the first electrode and the second electrode.

FIG. 18 shows a state in which the projection on the insulating chip surface rips the burnt piece of biological tissue as a result of the first electrode in FIG. 17 being drawn into the sheath.

FIG. 19 is a perspective view showing an example insulating chip having multiple linear projections on one surface.

FIG. 20 is a perspective view showing an example insulating chip having numerous pyramid-shaped projections on one surface.

FIG. 21 is a perspective view showing an example insulating chip having a spiral projection or groove on one surface.

FIG. 22 is a perspective view showing a hemispheric second electrode.

FIG. 23 is a perspective view showing a triangular plate-shaped second electrode.

FIG. 24 is a perspective view showing a second electrode bent in a direction intersecting the longitudinal direction of the first electrode.

FIG. 25 is a perspective view showing a state in which a burnt piece of biological tissue is stuck to the first electrode and the second electrode.

FIG. 26 is a perspective view showing a state in which a burnt piece of biological tissue is nipped between the second electrode and the insulating chip.

FIG. 27 is a perspective view showing a state in which a manipulation wire is twisted with the second electrode being pressed against the burnt piece of biological tissue.

FIG. 28 is perspective view showing a state in which the second electrode is rotated at high speed about the axis of the first electrode.

FIG. 29 is a plan view showing an example handle that has no sheath dial and rotates the knife part with respect to the sheath with an electrode dial.

FIG. 30 is a plan view showing an example handle that has no electrode dial and rotates the sheath with respect to the knife part with the sheath dial.

FIG. 31 is a vertical cross section of the handle, showing an example fixing mechanism.

### DESCRIPTION OF EMBODIMENTS

A high-frequency treatment tool according to an embodiment of the present invention and a method for manipulating a high-frequency treatment tool will be described below with reference to the drawings.

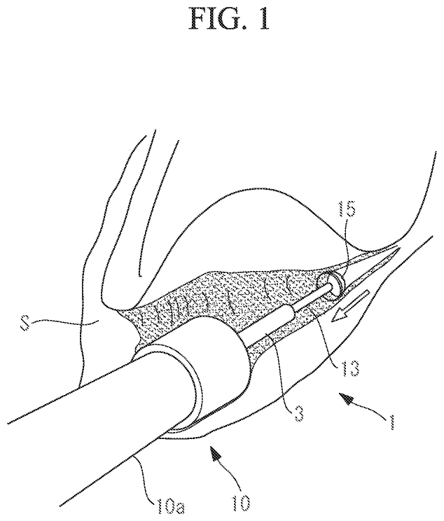

As shown in FIG. 1, for example, a high-frequency treatment tool 1 according to this embodiment is a treatment tool in which the distal end thereof is introduced into the body (living body) through a channel (not shown) provided in an insertion part 10a of an endoscope 10. Reference sign S in FIG. 1 indicates a lesion site in the body.

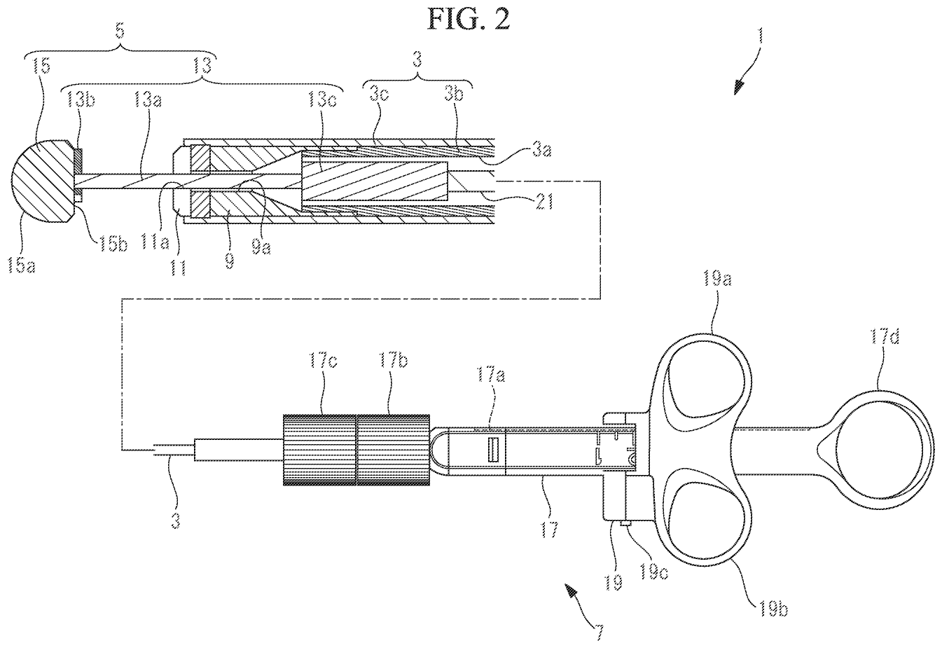

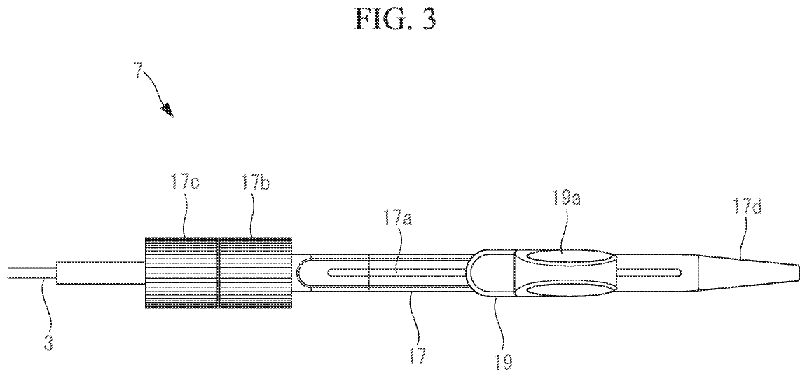

As shown in FIGS. 2 and 3, the high-frequency treatment tool 1 includes a thin, long, flexible cylindrical sheath 3 and a knife part 5 that is moved forward and backward at the distal end side of the sheath 3. Hereinbelow, the direction toward the distal end of the sheath 3 will be called the forward direction, and the direction toward the proximal end of the sheath 3 will be called the backward direction.

The sheath 3 is formed so as to be able to pass through the channel in the endoscope 10. The sheath 3 includes a cylindrical densely wound coil 3b having an inner hole 3a penetrating in the longitudinal direction, and a cylindrical insulating tube 3c covering the outer circumference of the densely wound coil 3b.

The densely wound coil 3b can easily change the shape thereof in accordance with a change in shape of the insertion part 10a of the endoscope 10 with the sheath 3 being inserted through the channel in the endoscope 10. The densely wound coil 3b can also transmit torque while maintaining flexibility.

The insulating tube 3c is formed of, for example, a heat-resistant flexible resin material, such as a tetrafluoroethylene material.

A tubular stopper member 9 having a through-hole 9a penetrating in the longitudinal direction of the sheath 3 and a ring-shaped insulating chip (cap member) 11 disposed closer to the distal end of the sheath 3 than the stopper member 9 is are provided at the distal end of the sheath 3.

The stopper member 9 is connected to the distal end of the densely wound coil 3b. The inner circumferential surface and the outer circumferential surface of the stopper member 9 and the densely wound coil 3b, respectively, are formed to be substantially flush with each other at the connected portion therebetween.

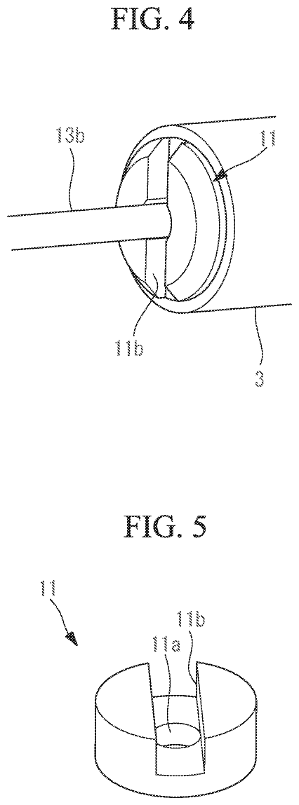

As shown in FIGS. 2 and 4, the insulating chip 11 is disposed at the distal end of the sheath 3, and the outer circumferential surface thereof is covered by the insulating tube 3c. The insulating chip 11 is formed of, for example, a heat-resistant, electrically insulating material, such as a ceramic material. The insulating chip 11 has a through-hole 11a penetrating in the longitudinal direction of the sheath 3. The through-hole 11a in the insulating chip 11 has substantially the same diameter as the through-hole 9a in the stopper member 9. In other words, the inner circumferential surface of the through-hole 11a in the insulating chip 11 is substantially flush with the inner circumferential surface of the through-hole 9a in the stopper member 9.

As shown in FIGS. 4 and 5, for example, the insulating chip 11 has, in one surface thereof, a groove (a recess and projections, a step) 11b recessed in the thickness direction of the insulating chip 11. The groove 11b extends straightly in the radial direction of the insulating chip 11 across the through-hole 11a. Furthermore, the groove 11b has substantially the same width as the diameter of the through-hole 11a. The insulating chip 11 is fixed to the distal end of the sheath 3 such that the surface having the groove 11b faces forward.

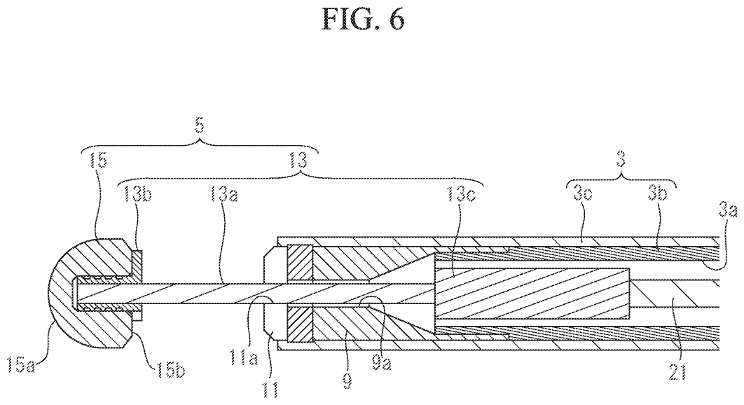

As shown in FIGS. 2 and 6, the knife part 5 includes an electrode part (rod-like member) 13 formed of a conducting material, and a hemispherical electrical insulator 15 fixed at the distal end of the electrode part 13.

The electrode part 13 includes a rod-like first electrode (first electrode member) 13a having a uniform diameter over the overall length thereof, a second electrode (second electrode member, electrode) 13b provided at the distal end of the first electrode 13a, and a stopper receiving part 13c provided at the proximal end of the first electrode 13a.

The first electrode 13a is provided through the through-hole 9a in the stopper member 9 and the through-hole 11a in the insulating chip 11 so as to be capable of projecting in the longitudinal direction from the distal end of the sheath 3. The first electrode 13a is formed of, for example, a conducting material, such as stainless steel. The proximal end of the first electrode 13a is electrically connected to the stopper receiving part 13c.

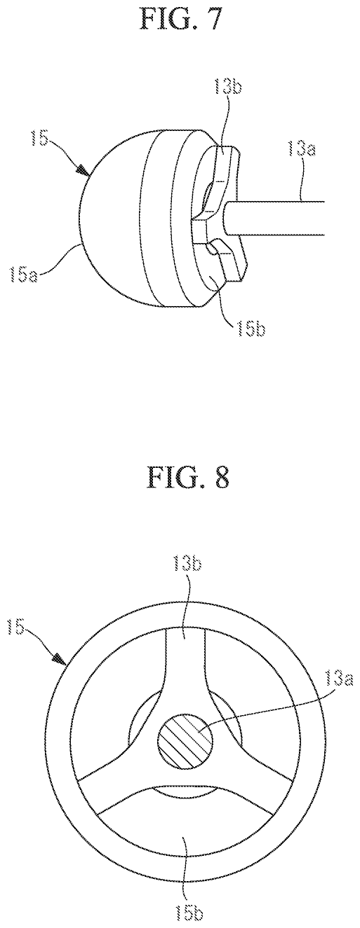

Similarly to the first electrode 13a, the second electrode 13b is formed of, for example, a conducting material, such as stainless steel, and is formed integrally with the distal end of the first electrode 13a. As shown in FIGS. 7 and 8, for example, the second electrode 13b extends radially in directions perpendicular to the axial direction of the first electrode 13a from the distal end of the first electrode 13a. In the example shown in FIGS. 7 and 8, the second electrode 13b extends radially in three directions about the axis of the first electrode 13a at equal intervals in the circumferential direction. The radially extending portions of the second electrode 13b each have, for example, a quadrate shape, such as a rectangular shape.

As shown in FIGS. 2 and 6, the stopper receiving part 13c is formed of a conducting material having a larger diameter than the first electrode 13a in cross section and has a cylindrical shape concentric with the first electrode 13a. When the electrode part 13 is moved forward to the maximum, the stopper receiving part 13c hits the proximal end of the stopper member 9 and restricts further forward movement of the electrode part 13.

The electrical insulator 15 is formed of a heat-resistant electrical insulator, such as a ceramic material. The electrical insulator 15 has substantially the same outside diameter as the outside diameter of the insulating chip 11. As shown in FIGS. 6 and 7, for example, the electrical insulator 15 is disposed such that a spherical part 15a faces forward and a planar part 15b faces backward. The second electrode 13b is fixed to the planar part 15b and extends radially along the planar part 15b.

The high-frequency treatment tool 1 also includes a handle 7 that is connected at the proximal end of the sheath 3 so that actuation of the handle 7 shifts the relative position between the sheath 3 and the knife part 5. The handle 7 is configured to manipulate the relative movement and rotation of the sheath 3 and the knife part 5. As shown in FIGS. 2 and 3, the handle 7 includes a handle body 17 having a longitudinal axis extending in the longitudinal direction of the sheath 3, a manipulation slider 19 provided so as to be movable in the direction parallel to the longitudinal axis of the handle body 17 with respect to the handle body 17, and a manipulation wire 21 formed of a conducting material and connecting the manipulation slider 19 and the knife part 5.

The handle body 17 includes a guide groove 17a extending straightly along the longitudinal axis, an electrode dial 17b formed of a cylindrical member connected to the manipulation wire 21, a sheath dial 17c formed of a cylindrical member connected to the densely wound coil 3b, and a finger ring 17d for a thumb of an operator. The finger ring 17d is disposed at the proximal end of the handle body 17.

The manipulation slider 19 is provided so as to be straightly movable along the guide groove 17a in the handle body 17. As shown in FIG. 2, the manipulation slider 19 includes a finger ring 19a for the index finger of the operator, a finger ring 19b for the middle finger of the operator, and a connecting connector part 19c to which a cord (not shown) leading to a high-frequency generator (not shown) and the manipulation wire 21 are electrically connected.

The finger ring 19a and the finger ring 19b are disposed at a distance from each other in the direction perpendicular to the longitudinal axis of the handle body 17. For example, by hooking the thumb of one hand in the finger ring 17d of the handle body 17 and hooking the index finger and the middle finger of the same hand in the finger ring 19a and the finger ring 19b of the manipulation slider 19, respectively, it is possible to easily move the manipulation slider 19 along the guide groove 17a with respect to the handle body 17 with only one hand.

As shown in FIG. 2, the manipulation wire 21 is disposed in the inner hole 3a of the sheath 3. The distal end of the manipulation wire 21 is connected to the stopper receiving part 13c of the knife part 5, and the proximal end of the manipulation wire 21 is electrically connected to the connecting connector part 19c of the manipulation slider 19. Hence, the electrode part 13 of the knife part 5 is electrically connected to the connecting connector part 19c of the manipulation slider 19 by the manipulation wire 21.

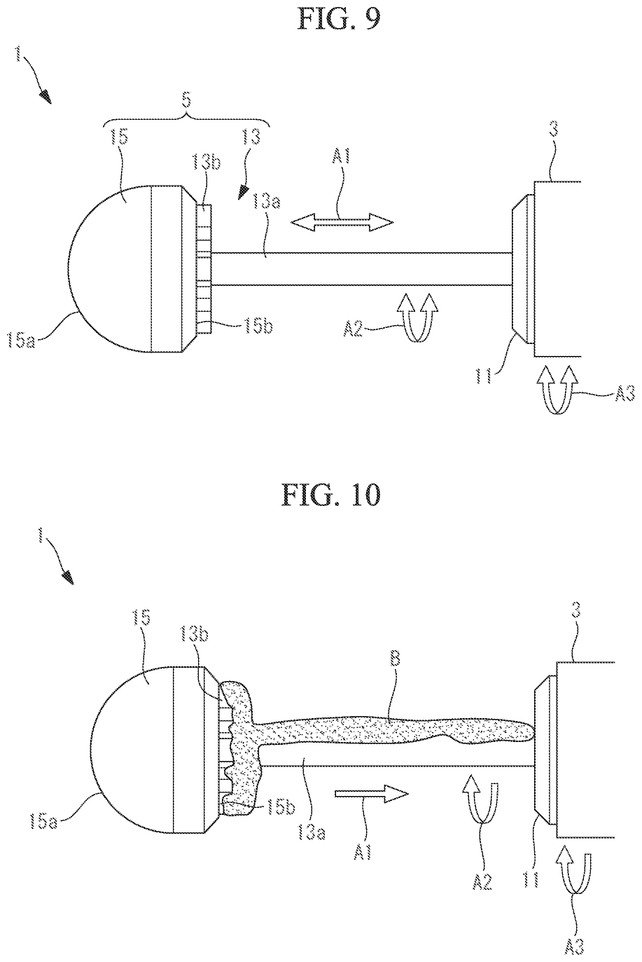

Furthermore, the manipulation wire 21 is provided so as to be movable in the longitudinal direction of the sheath 3, together with the manipulation slider 19. Accordingly, when the manipulation slider 19 is moved along the guide groove 17a in the handle body 17, the manipulation wire 21 is pushed or pulled in the longitudinal direction of the sheath 3, and thus, a pushing force or a pulling force is transmitted to the knife part 5. As a result, as shown by arrow A1 in FIG. 9, the knife part 5 moves in the longitudinal direction of the sheath 3 with respect to the sheath 3. In other words, the electrode part 13 of the knife part 5 is moved forward/backward with respect to the insulating chip 11 of the sheath 3 with the forward/backward motion of the manipulation wire 21.

The manipulation wire 21 may be formed either of a solid wire or a stranded wire. When the manipulation wire 21 is formed of a solid wire, it is possible to efficiently transmit the torque. The material of the manipulation wire 21 when formed of a solid wire is not specifically limited and may be stainless steel, such as SUS301, SUS302, SUS304, and SUS316, a nickel alloy, such as a Ni—Cr—Fe type, or piano wire, such as SWP-A.

When the manipulation wire 21 is formed of a stranded wire, it is possible to efficiently transmit the torque while maintaining flexibility. The structure of the stranded wire is not specifically limited and may be 1×7 strands and 1×19 strands. The material of the manipulation wire 21 when formed of a stranded wire is not specifically limited and may be stainless steel, such as SUS301, SUS302, SUS304, and SUS316, a nickel alloy, such as a Ni—Cr—Fe type, or piano wire, such as SWP-A.

The electrode dial 17b and the sheath dial 17c are provided on the front side of the handle body 17 with respect to the guide groove 17a and are disposed at positions shifted from each other in the longitudinal-axis direction of the handle body 17. The electrode dial 17b and the sheath dial 17c are provided so as to be independently rotatable about the longitudinal axis of the handle body 17. The operator, while gripping the finger ring 17d of the handle body 17 and the finger rings 19a and 19b of the manipulation slider 19 with one hand, can manipulate one of the electrode dial 17b and the sheath dial 17c with the other hand.

When the electrode dial 17b is rotated about the longitudinal axis of the handle body 17, the rotation about the longitudinal axis of the sheath 3 is transmitted to the knife part 5 through the manipulation wire 21. As a result, as shown by arrow A2 in FIG. 9, the knife part 5 rotates about the longitudinal axis of the sheath 3 with respect to the sheath 3 and the insulating chip 11. In contrast, when the sheath dial 17c is rotated about the longitudinal axis of the handle body 17, the rotation about the longitudinal axis of the sheath 3 is transmitted to the overall sheath 3 through the densely wound coil 3b. As a result, as shown by arrow A3 in FIG. 9, the insulating chip 11, together with the sheath 3, rotates about the longitudinal axis of the sheath 3 with respect to the knife part 5. Although a monopolar requires the insulating chip 11, a bipolar may use a cap with an electrode, instead of the insulating chip 11.

The motion of the thus-configured high-frequency treatment tool 1 will be described.

When the manipulation slider 19 is moved backward with respect to the handle body 17, the manipulation wire 21, together with the manipulation slider 19, moves backward with respect to the sheath 3. As a result, the first electrode 13a of the knife part 5 is drawn into the sheath 3 until the second electrode 13b of the knife part 5 hits the insulating chip 11 of the sheath 3.

In contrast, when the manipulation slider 19 is moved forward with respect to the handle body 17, the manipulation wire 21, together with the manipulation slider 19, moves forward with respect to the sheath 3. As a result, the first electrode 13a of the knife part 5 projects in the longitudinal direction from the distal end of the sheath 3 until the stopper receiving part 13c of the knife part 5 hits the stopper member 9 in the sheath 3.

Furthermore, when the electrode dial 17b is rotated about the longitudinal axis of the handle body 17, the knife part 5 rotates about the longitudinal axis of the sheath 3 with respect to the sheath 3 and the insulating chip 11. In contrast, when the sheath dial 17c is rotated about the longitudinal axis of the handle body 17, the insulating chip 11, together with the sheath 3, rotates about the longitudinal axis with respect to the knife part 5.

Next, the operation of the high-frequency treatment tool 1 according to this embodiment will be described below.

To endoscopically perform demucosation in the body by using the high-frequency treatment tool 1 according to this embodiment, first, an injection needle (not shown) is introduced into the body through the channel in the endoscope 10. Then, while viewing an endoscope image displayed on a monitor (not shown), physiological saline is injected into the submucosal layer at the site considered to be the lesion to be resected to swell the lesion site.

Next, a high-frequency knife (not shown) having a conventional needle-like electrode is introduced into the body through the channel in the endoscope 10 to perform initial cutting (precutting) for making a hole in one part of the mucous membrane around the lesion site. After the initial cutting (precutting) is performed, the high-frequency knife is removed from the channel.

Next, the tool is changed to the high-frequency treatment tool 1, and the sheath 3 is introduced into the body from the distal end thereof through the channel in the endoscope 10 with the knife part 5 being retracted to the maximum with the handle 7. When the distal end of the sheath 3 is projected from the distal end of the channel in the endoscope 10, the electrical insulator 15 disposed at the distal end of the sheath 3 enters the field of view of the endoscope 10, so, the operator performs treatment while viewing the image acquired by the endoscope 10 on the monitor.

In a state in which the knife part 5 is retracted to the maximum, only the electrical insulator 15 is exposed from the distal end of the sheath 3. Hence, the knife part 5 is not deeply inserted into the biological tissue. Furthermore, because the spherical part 15a of the spherical electrical insulator 15 is disposed so as to face forward, the biological tissue to which the electrical insulator 15 is in contact is not damaged.

Next, the knife part 5 is moved forward to the maximum with the handle 7. When the stopper receiving part 13c of the knife part 5 hits the stopper member 9 in the sheath 3, the forward movement of the knife part 5 is restricted, and the first electrode 13a and the second electrode 13b are exposed at the front side of the sheath 3. In this state, the knife part 5 is inserted into the hole that has been formed in advance in the initial cutting (precutting) from the electrical insulator 15.

Then, while a high-frequency current is supplied to the first electrode 13a and the second electrode 13b through the manipulation wire 21, the knife part 5 is moved in a predetermined cutting direction intersecting the longitudinal axis. For example, by hooking the portion from the distal end of the first electrode 13a to the second electrode 13b on the mucous membrane around the lesion site, the portion around the lesion site can be efficiently cut and cauterized.

Because the electrical insulator 15 provided at the distal end of the knife part 5 is formed of an insulating material, even though a high-frequency current is supplied to the first electrode 13a and the second electrode 13b, the biological tissue with which the electrical insulator 15 is in contact is not cut. Accordingly, it is possible to prevent an inconvenience whereby the operator unintentionally cuts the tissue in a deep layer, such as the muscle layer, with the electrical insulator 15.

In this case, when the biological tissue is cut and cauterized, for example, as shown in FIG. 10, a burnt piece B of the cut biological tissue sticks to the first electrode 13a and the second electrode 13b of the electrode part 13. When the burnt piece B of biological tissue sticks to at least one of the first electrode 13a and the second electrode 13b, the cutting performance with the electrode part 13 is degraded. Hence, the burnt piece B of biological tissue needs to be removed from the first electrode 13a and the second electrode 13b. Hereinbelow, the first electrode 13a and the second electrode 13b will be called simply “the electrodes 13a and 13b”.

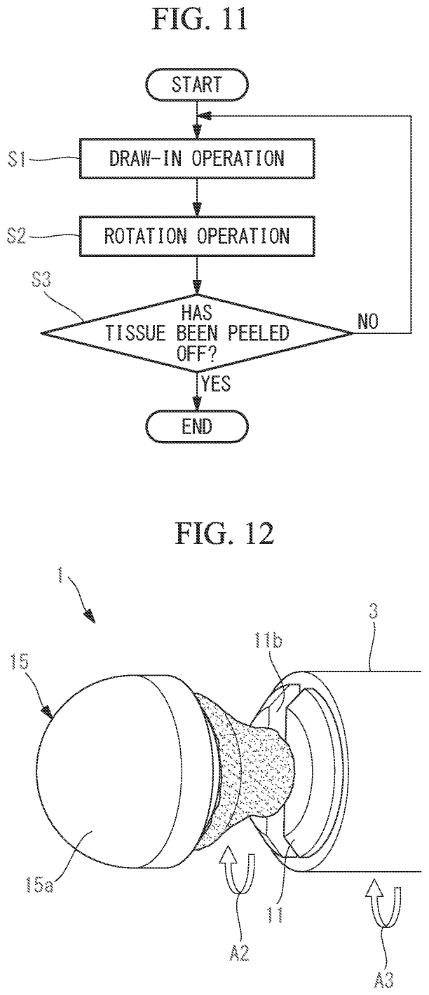

A method for manipulating a high-frequency treatment tool 1 when the burnt piece B of biological tissue is stuck to the electrodes 13a and 13b will be described below with reference to the flowchart in FIG. 11.

When the burnt piece B of biological tissue is stuck to the electrodes 13a and 13b, first, while the distal end of the sheath 3 remains inserted through the body through the channel in the endoscope 10, the knife part 5 is moved in a direction in which the first electrode 13a is drawn into the sheath 3 with the handle 7 (draw-in step S1), as shown by arrow A1 in FIG. 10.

As a result, as shown in FIG. 12, for example, the burnt piece B stuck to the first electrode 13a is accumulated between the second electrode 13b and the insulating chip 11 while being pushed by the surface of the insulating chip 11. The burnt piece B stuck to the second electrode 13b, together with the burnt piece B stuck to the first electrode 13a, is pressed against the insulating chip 11 at the distal end of the sheath 3.

The burnt piece B of the biological tissue pressed against the insulating chip 11 enters the groove 11b provided in the surface of the insulating chip 11. As a result, the burnt piece B of biological tissue is caught by the edge of the groove 11b, making it possible to increase the friction between the burnt piece B of biological tissue and the insulating chip 11.

Next, with the burnt piece B of biological tissue being pressed against the insulating chip 11 with the handle 7, the electrode part 13 and the sheath 3 are relatively rotated about the longitudinal axis of the sheath 3 (rotation step S2). For example, as shown by arrow A2 in FIG. 12, the knife part 5 is rotated about the longitudinal axis of the sheath 3 with respect to the sheath 3 with the electrode dial 17b. Alternatively, as shown by arrow A3 in FIG. 12, the sheath 3 is rotated about the longitudinal axis with respect to the knife part 5 with the sheath dial 17c. Alternatively, instead of rotating only one of the knife 5 and the sheath 3, the rotation direction of one of them may be switched, and the knife 5 and the sheath 3 may be simultaneously rotated. Note that, also when the knife 5 and the sheath 3 are rotated in the same direction, by differentiating the rotation speeds thereof, the electrode part 13 and the sheath 3 can be relatively rotated about the longitudinal axis of the sheath 3.

As a result, the friction between the burnt piece B of biological tissue and the insulating chip 11 causes a portion of the burnt piece B of biological tissue stuck to the electrodes 13a and 13b and a portion of the same biting into the insulating chip 11 to displace in opposite directions to each other about the longitudinal axis of the sheath 3, creating torsion in the burnt piece B of biological tissue and creating a shearing force in the burnt piece B. As a result, the burnt piece B of biological tissue peels off from the electrodes 13a and 13b. Then, when the torsion causes the burnt piece B of biological tissue to crack, the burnt piece B of biological tissue comes off from the electrodes 13a and 13b.

If the burnt piece B of biological tissue does not come off from the electrodes 13a and 13b (step S3, “NO”), steps S1 and S2 are repeated until the burnt piece B of biological tissue is removed from the electrodes 13a and 13b. For example, steps S1 and S2 may be repeated after the knife part 5 is temporarily moved forward. Furthermore, in step S2, the electrode part 13 and the sheath 3 may be relatively rotated about the longitudinal axis of the sheath 3 while the burnt piece B of biological tissue is more strongly pressed against the insulating chip 11.

When the burnt piece B of biological tissue comes off from the electrodes 13a and 13b (step S3 “YES”), the process of removing the burnt piece B of biological tissue is finished. In this case, the knife part 5 is again moved forward to the maximum with the handle 7 to restart the treatment.

As has been described above, with the high-frequency treatment tool 1 according to this embodiment, when the burnt piece B of biological tissue is stuck to the electrodes 13a and 13b, simply by relatively moving and rotating the electrode part 13 and the sheath 3 with the handle 7, it is possible to remove the burnt piece B of biological tissue from the electrodes 13a and 13b while the sheath 3, the electrode part 13, and the like remain inserted through the channel in the endoscope 10. Accordingly, even when the burnt piece B of biological tissue is stuck to the electrodes 13a and 13b, it is possible to save the effort of removing the high-frequency treatment tool 1 from the channel in the endoscope 10 and to improve the work efficiency.

Furthermore, by providing the step, such as the groove 11b, in the surface of the insulating chip 11, the friction between the burnt piece B of biological tissue and the insulating chip 11 increases. Accordingly, when the electrode part 13 and the sheath 3 are relatively rotated about the longitudinal axis of the sheath 3 with the burnt piece B of biological tissue being pressed against the insulating chip 11, the burnt piece B of biological tissue is caught by the surface of the insulating chip 11, and torsion is more likely to be created in the burnt piece B of biological tissue. This makes it possible to efficiently remove the burnt piece B of biological tissue from the electrodes 13a and 13b.

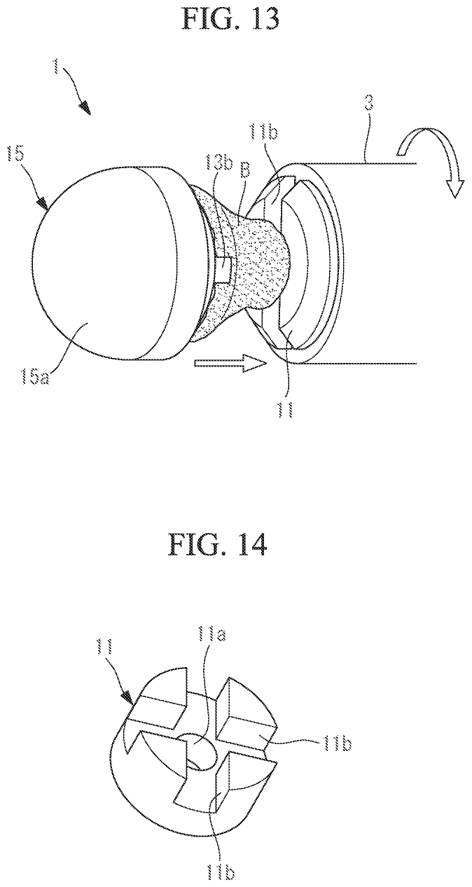

Furthermore, because the second electrode 13b extends in the direction intersecting the center axis of the first electrode 13a, for example, as shown in FIG. 13, when the electrode part 13 and the sheath 3 are relatively rotated about the longitudinal axis of the sheath 3 with the burnt piece B of biological tissue being pressed against the insulating chip 11, the burnt piece B of biological tissue is caught by the second electrode 13b. Accordingly, it is possible to displace a portion of the burnt piece B of biological tissue pressed against the insulating chip 11 and a portion of the same stuck to the second electrode 13b in opposite directions to each other about the longitudinal axis of the sheath 3. This makes it possible to prevent only the electrode part 13 from freely rotating while pressing the burnt piece B of biological tissue against the insulating chip 11 and to more reliably create torsion in the burnt piece B of biological tissue.

The first electrode 13a and the second electrode 13b may be formed of separate members, and the second electrode 13b may be fixed to the distal end of the first electrode 13a. This configuration makes machining easier, compared with a case where the first electrode 13a and the second electrode 13b are formed as a single member.

A modification of this embodiment will be described below.

In this embodiment, as an example in which a step is formed in one surface of the insulating chip 11, the insulating chip 11 has, in the surface thereof, one groove 11b extending in the radial direction. Instead of this, for example, as shown in FIG. 14, the insulating chip 11 may have multiple grooves 11b extending radially from the through-hole 11a across the through-hole 11a. Increasing the number of the grooves 11b can increase the friction between the burnt piece B of biological tissue and the insulating chip 11. Preferably, the grooves 11b are straight but are not limited to straight.

Alternatively, for example, the insulating chip 11 may have, on the surface thereof, a projection (step) projecting in the longitudinal direction of the through-hole 11a, instead of the groove 11b. With this configuration, the projection on the surface of the insulating chip 11 bites into the burnt piece B of biological tissue pressed against the surface of the insulating chip 11. Thus, the friction between the burnt piece B of biological tissue and the insulating chip 11 can be increased with a simple configuration.

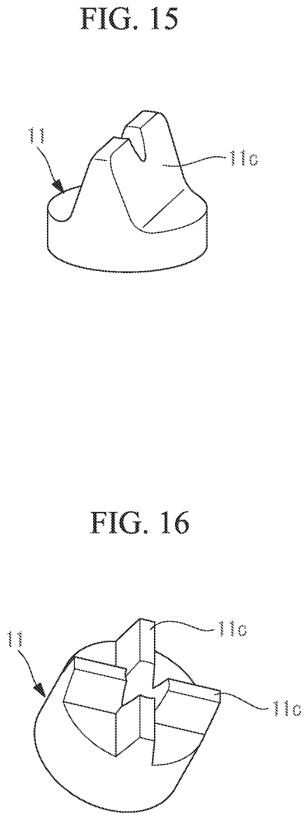

In this case, for example, as shown in FIG. 15, the insulating chip 11 may have one projection (step) 11c extending in the radial direction across the through-hole 11a. More specifically, the through-hole 11a is formed in the middle of the projection 11c. Note that the number of projections 11c does not necessarily have to be one, and as shown in FIG. 15, there may be a pair of projections 11c independently disposed at positions away from each other in the radial direction of the first electrode (rod-like member) 13a. In that case, the through-hole 11a may be formed at a position between the pair of projections 11c.

Furthermore, for example, as shown in FIG. 16, the insulating chip 11 may have multiple projections 11c extending radially from the through-hole 11a. Also in that case, there may be two pairs of projections 11c that are independently disposed at positions away from each other in the radial direction of the first electrode (rod-like member) 13a. Preferably, the projections 11c are straight but are not limited to straight.

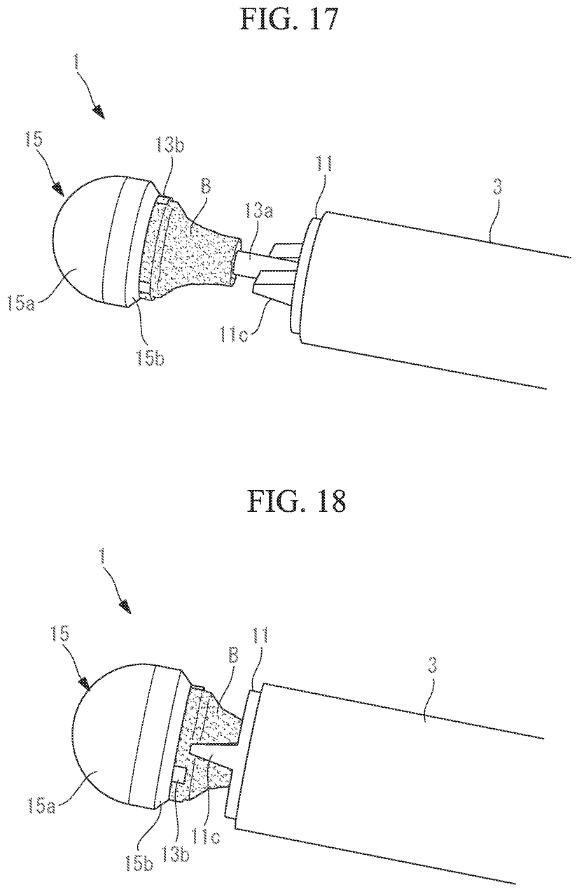

The insulating chip 11 having the projections 11c as shown in FIGS. 15 and 16 provides the following advantage. Specifically, not only it is possible to rip the burnt piece B of biological tissue stuck to the electrodes 13a and 13b with a shearing force created by the relative rotational motion between the insulating chip 11 and the knife part 5, but also, for example, as shown in FIGS. 17 and 18, it is possible to easily rip the burnt piece B of biological tissue stuck to the first electrode 13a with the motion of drawing the knife part 5 into the sheath 3. FIGS. 17 and 18 show an example case where the insulating chip 11 has two projections (steps) 11c extending in the radial direction across the through-hole 11a.

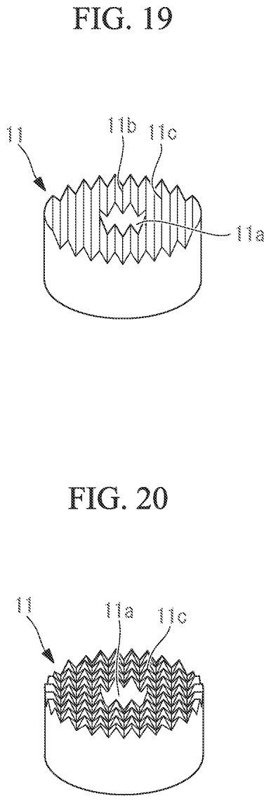

Furthermore, for example, as shown in FIG. 19, the insulating chip 11 may have, in the surface thereof, multiple linear sharp grooves 11b or projections 11c that are arranged at a small pitch. Furthermore, for example, as shown in FIG. 20, the insulating chip 11 may have numerous conical or pyramid-shaped projections 11c on the surface thereof.

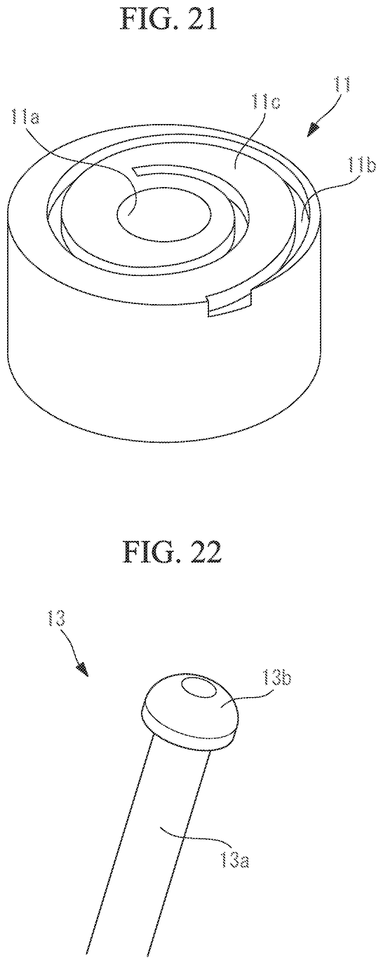

Furthermore, for example, as shown in FIG. 21, the insulating chip 11 may have a spiral groove 11b or projection 11c extending around the opening of the through-hole 11a in the surface thereof.

With this configuration, when the electrode part 13 and the sheath 3 are relatively rotated about the longitudinal axis of the sheath 3, the burnt piece B of biological tissue entrapped in the helical groove 11b or the projection 11c in the surface of the insulating chip 11 is easily removed in the radially outward direction with the rotational motion thereof.

In this embodiment, although the second electrode 13b extending radially in three directions from the first electrode 13a has been described as an example, the second electrode 13b only needs to have a shape extending radially in the direction perpendicular to the axial direction of the first electrode 13a. For example, the second electrode 13b may be either hemispherical, as shown in FIG. 22, or discoidal.

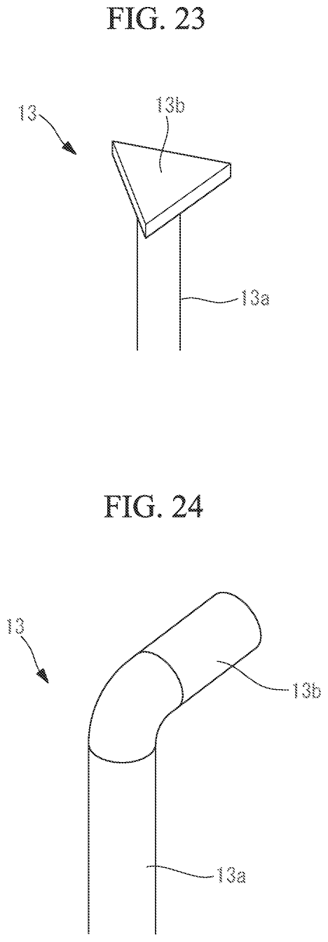

Furthermore, for example, the second electrode 13b may have a triangular plate shape extending in the radially outward direction of the first electrode 13a, as shown in FIG. 23, or, for example, a shape bent in a direction intersecting the longitudinal direction of the first electrode 13a, as shown in FIG. 24. Furthermore, the second electrode 13b may have an arbitrary shape having portions projecting in the radial direction and portions recessed in the radial direction that are arranged alternately in the circumferential direction, such as a polygonal shape having four or more corners, a star shape, and an elliptical shape. In the case where the second electrode 13b is any of these non-circular shapes, the burnt piece B of biological tissue is caught by the second electrode 13b, and torsion is more likely to be created in the burnt piece B of biological tissue.

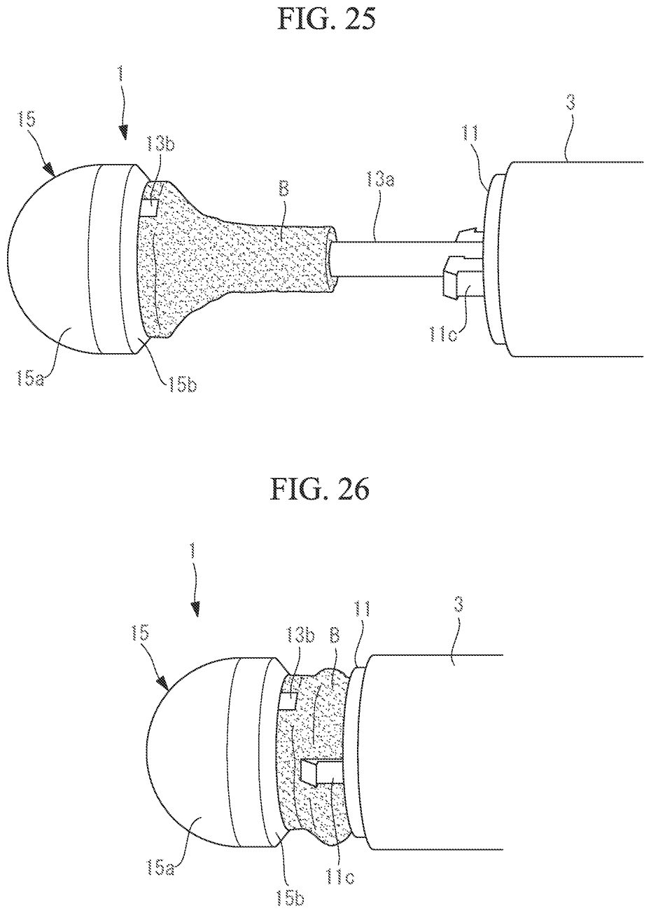

Furthermore, in this embodiment, when the burnt piece B of biological tissue stuck to the electrodes 13a and 13b is removed, the electrodes 13a and 13b may be rotated at high speed. For example, as shown in FIG. 25, when the burnt piece B of biological tissue is stuck to the electrodes 13a and 13b, first, the knife part 5 is drawn into the sheath 3 to nip the burnt piece B of biological tissue between the second electrode 13b and the insulating chip 11, as shown in FIG. 26. By doing so, the second electrode 13b is pressed against the burnt piece B of biological tissue.

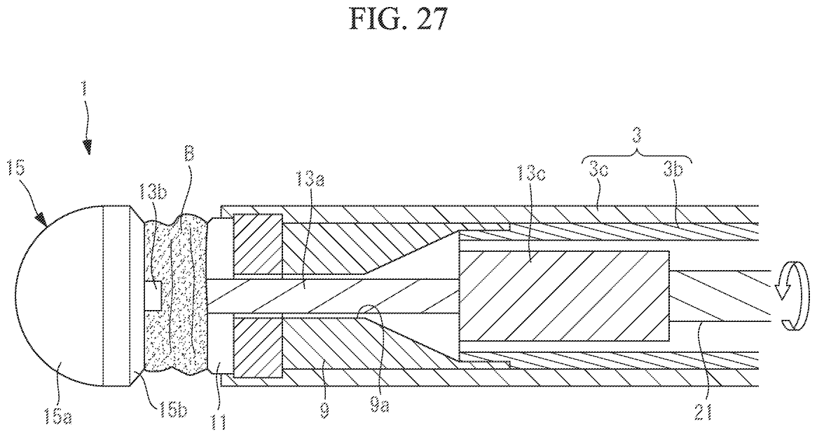

Next, as shown in FIG. 27, the manipulation wire 21 is twisted by rotating the electrode dial 17b around the longitudinal axis of the handle body 17 (twisting step). Because there is friction between the second electrode 13b and the burnt piece B of biological tissue, the second electrode 13b does not rotate, and the manipulation wire 21 accumulates the strain energy.

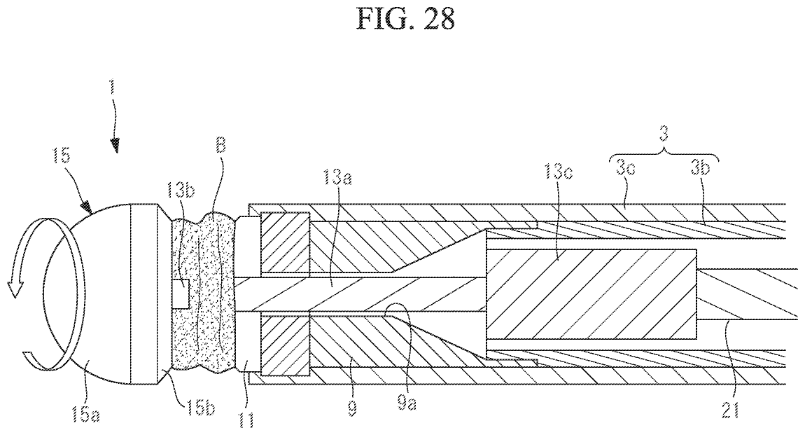

When the manipulation wire 21 has accumulated the strain energy, and the friction between the second electrode 13b and the burnt piece B of biological tissue becomes unable to stop the rotation of the second electrode 13b, in other words, when the torque acting on the second electrode 13b has become larger than the friction, as shown in FIG. 28, the second electrode 13b rotates at high speed about the axis of the first electrode 13a, applying a centrifugal force to the burnt piece B of biological tissue. This makes it possible to blow off the burnt piece B of biological tissue stuck to the second electrode 13b in the radially outward direction and, thus, to remove the burnt piece B from the second electrode 13b.

Furthermore, as shown in FIG. 27, when the strain energy becomes accumulated in the manipulation wire 21 by twisting the manipulation wire 21, the friction between the second electrode 13b and the burnt piece B of biological tissue may be reduced by moving the manipulation slider 19 toward the distal end. When the torque acting on the second electrode 13b becomes larger than the friction as a result of this, as shown in FIG. 28, the second electrode 13b rotates at high speed about the axis of the first electrode 13a. Also in this case, a centrifugal force is applied to the burnt piece B of biological tissue, and thus, it is possible to blow off the burnt piece B of biological tissue in the radially outward direction and, thus, to remove the burnt piece B from the second electrode 13b.

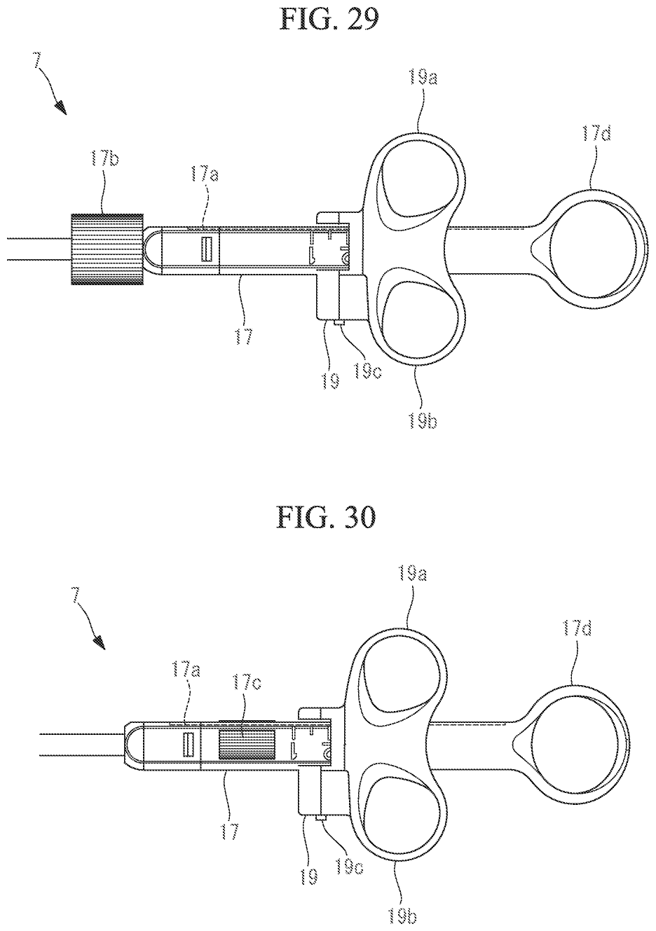

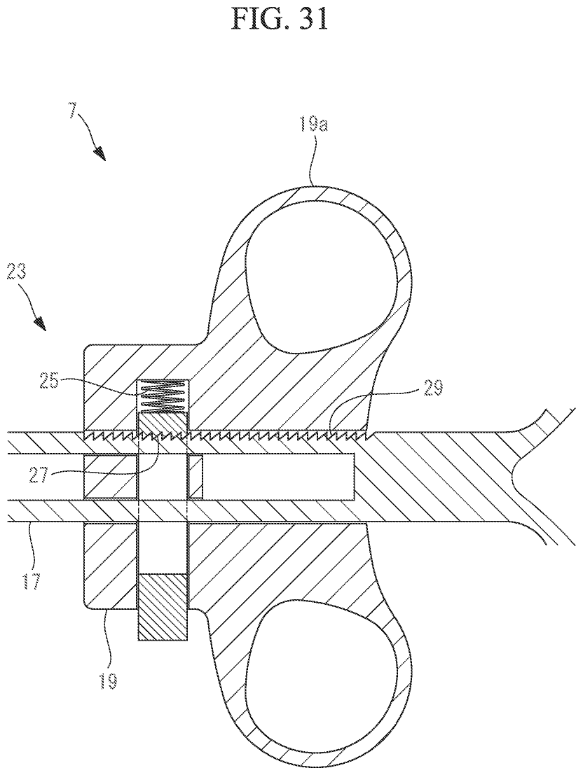

Furthermore, in this embodiment, the handle body 17 has both the electrode dial 17b and the sheath dial 17c. Instead of this, for example, as shown in FIG. 29, the handle body 17 may have no sheath dial 17c, and the knife part 5 may be rotated with respect to the sheath 3 with the electrode dial 17b. Furthermore, for example, as shown in FIG. 30, the handle body 17 may have no electrode dial 17b, and the sheath 3 may be rotated with respect to the knife part 5 with the sheath dial 17c. In that case, as shown in FIG. 30, the sheath dial 17c may be disposed in the handle body 17.

Furthermore, in this embodiment, the handle 7 may have a fixing mechanism 23, as shown in FIG. 31, for maintaining, for example, a state in which the manipulation wire 21 is taut, that is, the electrode part 3 is drawn into the sheath 3.

The fixing mechanism 23 is a ratchet mechanism provided on the manipulation slider 19 and includes a spring 25 and an engaging part (pawl) 27. The handle body 17 is provided with an engaged part (ratchet teeth) 29. The fixing mechanism 23 allows the manipulation slider 19 to move backward in the longitudinal axis of the handle body 17 with respect to the handle body 17 but does not allow the manipulation slider 19 to move forward.

The engaging part 27 of the fixing mechanism 23 is meshed with the engaged part 29 of the handle body 17 due to the restoring force of the spring 25. When the engaging part 27 and the engaged part 29 are meshed with each other, the manipulation slider 19 cannot move forward with respect to the handle body 17. However, even when the engaging part 27 and the engaged part 29 are meshed with each other, the manipulation slider 10 can move backward with respect to the handle body 17. This configuration makes it possible to maintain the state in which the first electrode 13a is drawn into the sheath 3.

Although the densely wound coil 3b has been described as an example in this embodiment, instead of this, for example, a component including a densely wound coil and a blade may be employed, or a multi-layer multi-thread coil may be employed. Both in the case where a component including a densely wound coil and a blade is employed and in the case where the multi-layer multi-thread coil is employed, it is possible to efficiently transmit the torque while maintaining flexibility. Furthermore, when only the knife part 5 is rotated, the sheath 3 only needs to be flexible and thus may be a resin tube.

Furthermore, in this embodiment, a liquid delivery means for discharging a liquid from the distal end of the sheath 3 through the inner hole 3a of the sheath 3 may be provided. In that case, a connecting port (not shown) communicating with the inner hole 3a of the sheath 3 may be provided in the handle body 17, and a syringe, a pump, and the like to be connected to the connecting port may be employed as the liquid delivery means.

By discharging the liquid from the distal end of the sheath 3 with the liquid delivery means, it is possible to spray the liquid onto the burnt piece B of biological tissue stuck to the electrode part 13. As a result, the burnt piece B of biological tissue is softened by the liquid, and the adhesion between the burnt piece B of biological tissue and the electrode part 13 decreases. Hence, by twisting the burnt piece B of biological tissue and softening the burnt piece B of biological tissue by means of liquid delivery, it is possible to more efficiently remove the burnt piece B of biological tissue.

The following aspects can be also derived from the embodiments.

A first aspect of the present invention is a high-frequency treatment tool including: a tubular sheath; a cap member fixed to a distal end of the sheath; and an electrode part projecting in a longitudinal direction of the sheath through the cap member and provided so as to be relatively movable in the longitudinal direction and relatively rotatable about a longitudinal axis of the sheath with respect to the cap member. The electrode part includes a first electrode member extending in the longitudinal direction and at least one second electrode member extending from a distal end of the first electrode member in a direction intersecting the longitudinal direction. The cap member has at least one step in a direction facing the second electrode member. The electrode part and the sheath are relatively moved in the longitudinal direction and are relatively rotated about the longitudinal axis by performing manipulation at a proximal end of the sheath.

According to this aspect, it is possible to cut and cauterize biological tissue by bringing the electrode part energized with a high-frequency current into contact with the biological tissue. For example, by hooking a portion from the distal end of the first electrode member to the second electrode member of the electrode part on biological tissue, such as a mucous membrane, it is possible to efficiently cut and cauterize the biological tissue.

When a burnt piece of biological tissue is stuck to the first electrode member and the second electrode member of the electrode part as a result of cutting and cauterizing the biological tissue, first, the burnt piece of biological tissue stuck to the electrode members is pressed against the cap member by relatively moving the electrode part and the sheath in a direction in which the electrode part is drawn into the sheath. As a result of the burnt piece of biological tissue being caught by the step on the surface of the cap member, the friction between the burnt piece of biological tissue and the cap member increases. Next, by relatively rotating the electrode part and the sheath about the longitudinal axis of the sheath while the burnt piece of biological tissue is pressed against the cap member, the friction between the burnt piece of biological tissue and the cap member causes torsion in the burnt piece of biological tissue. As a result, the burnt piece of biological tissue is cracked, and the burnt piece of biological tissue comes off from the first electrode member and the second electrode member.

Accordingly, when a burnt piece of biological tissue is stuck to the electrode part while treatment is performed in the living body through the endoscope channel, simply by relatively moving and rotating the electrode part and the sheath, it is possible to remove the burnt piece of biological tissue from the electrode part while the sheath, the electrode part, and the like remain inserted through the endoscope channel. This saves the effort of removing the high-frequency treatment tool from the endoscope channel when a burnt piece of biological tissue is stuck to the electrode part and improves the work efficiency.

The high-frequency treatment tool according to this aspect may further include a handle that manipulates a relative movement between the sheath and the electrode part at the proximal end of the sheath.

In this aspect, the handle may include a handle body connected to the sheath and having an axis extending in the longitudinal direction of the sheath, and a manipulation slider connected to the electrode part and movable along the axis of the handle body with respect to the handle body.

This configuration makes it possible to relatively move the electrode part and the sheath in the longitudinal direction of the sheath simply by moving the manipulation slider along the axis of the handle body with respect to the handle body.

In the high-frequency treatment tool according to this aspect, the handle may include an electrode dial connected to the electrode part and rotatable about an axis extending in the longitudinal direction of the sheath, and a sheath dial connected to the sheath and rotatable about the axis.

This configuration makes it possible to rotate the electrode part about the longitudinal axis of the sheath with respect to the sheath by rotating the electrode dial about the axis extending in the longitudinal direction of the sheath. Furthermore, it is possible to rotate the sheath about the longitudinal axis of the sheath with respect to the electrode part by rotating the sheath dial about the axis extending in the longitudinal direction of the sheath.

In the high-frequency treatment tool according to this aspect, the step may be at least one groove extending in a direction intersecting a longitudinal axis of the first electrode member.

With this configuration, the burnt piece of biological tissue pressed against the cap member enters the groove in the surface of the cap member. Thus, it is possible to improve the friction between the burnt piece of biological tissue and the cap member with a simple configuration.

In this aspect, the cap member may have a through-hole penetrating in a direction of the longitudinal axis of the first electrode member, the first electrode member may pass through the through-hole, and the groove may extend across the through-hole.

In the high-frequency treatment tool according to this aspect, the step may be at least one projection projecting in a direction of a longitudinal axis of the first electrode member.

With this configuration, the projection on the surface of the cap member bites into the burnt piece of biological tissue pressed against the cap member. Thus, it is possible to improve the friction between the burnt piece of biological tissue and the cap member with a simple configuration.

In this aspect, the cap member may have a through-hole penetrating in the direction of the longitudinal axis of the first electrode member, the first electrode member may pass through the through-hole, the projection may include a pair of projections formed at positions away from each other in a radial direction of the first electrode member, the pair of projections may extend in the radial direction of the first electrode member, and the through-hole may be formed between the pair of projections.

In the high-frequency treatment tool according to this aspect, the first electrode member and the second electrode member may be formed of separate members and fixed to each other.

This configuration makes machining easy compared with a case where the first electrode member and the second electrode member are formed as a single member.

A second aspect of the present invention is a high-frequency treatment tool including: a tubular sheath; a cap member fixed to a distal end of the sheath; and a rod-like member projecting in a longitudinal direction of the sheath through the cap member and provided so as to be relatively movable in the longitudinal direction and relatively rotatable about a longitudinal axis of the sheath with respect to the cap member. The rod-like member includes, at a distal end thereof, at least one electrode extending in a direction intersecting a longitudinal axis of the rod-like member. The cap member has at least one step in a direction facing the electrode. The rod-like member and the sheath are relatively moved in the longitudinal direction and are relatively rotated about the longitudinal axis by performing manipulation at a proximal end of the sheath.

The high-frequency treatment tool according to this aspect may further include a handle that manipulates a relative movement between the sheath and the rod-like member at the proximal end of the sheath.

In the high-frequency treatment tool according to this aspect, the handle may include a handle body connected to the sheath and having an axis extending in the longitudinal direction of the sheath, and a manipulation slider connected to the rod-like member and movable along an axis of the handle body with respect to the handle body.

In the high-frequency treatment tool according to this aspect, the handle may include an electrode dial connected to the rod-like member and rotatable about an axis extending in the longitudinal direction of the sheath, and a sheath dial connected to the sheath and rotatable about the axis.

In the high-frequency treatment tool according to this aspect, the step may be at least one groove extending in a direction intersecting the longitudinal axis of the rod-like member.

In this aspect, the cap member may have a through-hole penetrating in a direction of the longitudinal axis of the rod-like member, the rod-like member may pass through the through-hole, and the groove may extend across the through-hole.

In the high-frequency treatment tool according to this aspect, the step may be at least one projection projecting in a direction of the longitudinal axis of the rod-like member.

In this aspect, the cap member may have a through-hole penetrating in the direction of the longitudinal axis of the rod-like member, the rod-like member may pass through the through-hole, the projection may include a pair of projections formed at positions away from each other in a radial direction of the rod-like member, the pair of projections may extend in the radial direction of the rod-like member, and the through-hole may be formed between the pair of projections.

A third aspect of the present invention is a method for manipulating a high-frequency treatment tool, the method including: a draw-in step in which, in a state in which the high-frequency treatment tool including an electrode part projecting in a longitudinal direction of a sheath through a cap member fixed to a distal end of the sheath is inserted through a living body, the electrode part and the sheath are relatively moved in a direction in which the electrode part is drawn into the sheath to press biological tissue stuck to the electrode part against the cap member; and a rotation step in which the electrode part and the cap member are relatively rotated about the longitudinal axis of the sheath while pressing the biological tissue against the cap member to separate the biological tissue from the electrode part.

According to this aspect, as a result of the electrode part and the sheath being relatively moved in the direction in which the electrode part is drawn into the sheath in the draw-in step while the high-frequency treatment tool remains inserted through the living body, the biological tissue stuck to the electrode part is pressed against the cap member. Then, as a result of the electrode part and the cap member being relatively rotated about the longitudinal axis of the sheath while pressing the biological tissue against the cap member in the rotation step, the friction between the biological tissue and the cap member causes torsion in the biological tissue. As a result, the biological tissue is cracked and comes off from the electrode part.

Accordingly, when a burnt piece of biological tissue is stuck to the electrode part while treatment is performed in the living body through the endoscope channel, it is possible to remove the burnt piece of biological tissue from the electrode part while the high-frequency treatment tool remains inserted through the endoscope channel with a simple method in which simply the electrode part and the sheath are relatively moved and rotated.

In the method for manipulating the high-frequency treatment tool according to this aspect, in the rotation step, the electrode part and the cap member may be relatively rotated about the longitudinal axis of the sheath while pressing the biological tissue against the cap member to create torsion in the biological tissue stuck to the electrode part.

In the method for manipulating the high-frequency treatment tool according to this aspect, the cap member may have a projection projecting toward the electrode part, and the draw-in step may include pressing the biological tissue against the cap member to rip the biological tissue with the projection.

The method for manipulating the high-frequency treatment tool according to this aspect may further include a twisting step in which a wire for transmitting a rotational force about the longitudinal axis of the sheath to the electrode part is twisted with the biological tissue being pressed against the cap member in the draw-in step. The rotation step may include releasing a torque created by the wire twisted in the twisting step and acting on the electrode part to rotate the electrode part about the longitudinal axis of the sheath with respect to the cap member.

### REFERENCE SIGNS LIST

1 high-frequency treatment tool

3 sheath

7 handle

11 insulating chip (cap member)

11a through-hole

11b groove (step)

11c projection (step)

13 electrode part (rod-like member)

13a first electrode (first electrode member)

13b second electrode (second electrode member, electrode)

17 handle body

17b electrode dial

17c sheath dial

19 manipulation slider

S1 draw-in step

S2 rotation step

1. A high-frequency treatment tool comprising:
a tubular sheath;
a cap member fixed to a distal end of the sheath; and
an electrode part provided so as to be relatively movable in a longitudinal direction of the sheath and relatively rotatable about a longitudinal axis of the sheath with respect to the cap member, wherein
the electrode part and the cap member are relatively rotated about the longitudinal axis of the sheath while pressing biological tissue stuck to the electrode part against the cap member to separate the biological tissue from the electrode part.

2. The high-frequency treatment tool according to claim 1, further comprising a handle that is connected at a proximal end of the tubular sheath so that actuation of the handle shifts a relative position between the sheath and the electrode part.

3. The high-frequency treatment tool according to claim 2, wherein the handle includes an electrode dial connected to the electrode part so that the electrode dial is rotatable about an axis extending in the longitudinal direction of the sheath, and a sheath dial connected to the sheath so that the sheath dial is rotatable about the axis.

4. The high-frequency treatment tool according to claim 1, wherein
the electrode part includes a first electrode member extending in the longitudinal direction and at least one second electrode member extending from a distal end of the first electrode member in a direction intersecting the longitudinal direction,
the cap member has at least one step in a direction facing the second electrode member, and
the step is at least one groove extending in a direction intersecting a longitudinal axis of the first electrode member.

5. The high-frequency treatment tool according to claim 4, wherein
the cap member has a through-hole penetrating in a direction of the longitudinal axis of the first electrode member,
the first electrode member passes through the through-hole, and
the groove extends across the through-hole.

6. The high-frequency treatment tool according to claim 1, wherein
the electrode part includes a first electrode member extending in the longitudinal direction and at least one second electrode member extending from a distal end of the first electrode member in a direction intersecting the longitudinal direction,
the cap member has at least one step in a direction facing the second electrode member, and
the step is at least one projection projecting in a direction of a longitudinal axis of the first electrode member.

7. The high-frequency treatment tool according to claim 1, wherein
the electrode part includes a first electrode member extending in the longitudinal direction and at least one second electrode member extending from a distal end of the first electrode member in a direction intersecting the longitudinal direction,
the cap member has at least one step in a direction facing the second electrode member, and
the step is a groove extending straightly in a direction intersecting a longitudinal axis of the first electrode member.

8. The high-frequency treatment tool according to claim 6, wherein
the cap member has a through-hole penetrating in the direction of the longitudinal axis of the first electrode member,
the first electrode member passes through the through-hole,
the projection includes a pair of projections formed at positions away from each other in a radial direction of the first electrode member,
the pair of projections extend in the radial direction of the first electrode member, and
the through-hole is formed between the pair of projections.

9. A high-frequency treatment tool comprising:
a tubular sheath;
a cap member fixed to a distal end of the sheath; and
a rod-like member extending along a longitudinal axis of the sheath through the cap member and provided so as to be relatively movable along the longitudinal axis of the sheath and relatively rotatable about the longitudinal axis of the sheath with respect to the cap member, wherein
the rod-like member includes, at a distal end thereof, at least one electrode extending in a direction intersecting a longitudinal axis of the rod-like member,
the cap member has at least one step in a direction facing the electrode,
the step extends in the direction intersecting the longitudinal axis of the rod-like member, and
the rod-like member and the sheath are configured to relatively move along the longitudinal axis of the sheath and are configured to relatively rotate about the longitudinal axis of the sheath.

10. The high-frequency treatment tool according to claim 9, further comprising a handle that is connected at a proximal end of the tubular sheath so that actuation of the handle shifts a relative position between the sheath and the electrode.

11. The high-frequency treatment tool according to claim 10, wherein the handle includes an electrode dial connected to the rod-like member so that the electrode dial is rotatable about an axis extending in the longitudinal direction of the sheath, and a sheath dial connected to the sheath so that the sheath dial is rotatable about the axis.

12. The high-frequency treatment tool according to claim 9, wherein the step is at least one groove.

13. The high-frequency treatment tool according to claim 12, wherein
the cap member has a through-hole penetrating in a direction of the longitudinal axis of the rod-like member,
the rod-like member passes through the through-hole, and
the groove extends across the through-hole.

14. The high-frequency treatment tool according to claim 9, wherein the step is at least one projection projecting in a direction of the longitudinal axis of the rod-like member.

15. The high-frequency treatment tool according to claim 14, wherein
the cap member has a through-hole penetrating in the direction of the longitudinal axis of the rod-like member,
the rod-like member passes through the through-hole,
the projection includes a pair of projections formed at positions away from each other in a radial direction of the rod-like member,
the pair of projections extend in the radial direction of the rod-like member, and
the through-hole is formed between the pair of projections.

16. The high-frequency treatment tool according to claim 9, wherein the step is a groove extending straightly in the direction intersecting the longitudinal axis of the rod-like member.

17. A method for manipulating a high-frequency treatment tool, the method comprising:
in a state in which the high-frequency treatment tool including an electrode part projecting in a longitudinal direction of a sheath through a cap member fixed to a distal end of the sheath is inserted through a living body, relatively moving the electrode part and the sheath in a direction in which the electrode part is drawn into the sheath to press biological tissue stuck to the electrode part against the cap member; and
relatively rotating the electrode part and the cap member about a longitudinal axis of the sheath while pressing the biological tissue against the cap member to separate the biological tissue from the electrode part.

18. The method for manipulating the high-frequency treatment tool according to claim 17, wherein, in the relatively rotating, relatively rotating the electrode part and the cap member about the longitudinal axis of the sheath while pressing the biological tissue against the cap member to cause torsion in the biological tissue.

19. The method for manipulating the high-frequency treatment tool according to claim 17, wherein
the cap member has a projection projecting toward the electrode part, and
the relatively moving includes pressing the biological tissue against the cap member to rip the biological tissue with the projection.

20. The method for manipulating the high-frequency treatment tool according to claim 17, further comprising twisting a wire for transmitting a rotational force about the longitudinal axis of the sheath to the electrode part with the biological tissue being pressed against the cap member in the relatively moving,
wherein the relatively rotating includes releasing a torque created by the wire twisted in the twisting and acting on the electrode part to rotate the electrode part about the longitudinal axis of the sheath with respect to the cap member.
